(19) United States
(12) Patent Application Publication
(10) Pub. No.: US 2026/0228967 A1
(43) Pub. Date: Aug. 6, 2026

(54) METHOD AND DEVICE FOR GENERATING THREE-DIMENSIONAL TOOTH MODEL

(71) Applicant: KOREA UNIVERSITY RESEARCH AND BUSINESS FOUNDATION, Seoul (KR)

(72) Inventors: In-Seok SONG, Seoul (KR); Seung Jun BAEK, Seoul (KR); Sae Hyun KIM, Incheon (KR)

(21) Appl. No.: 19/153,193

(22) PCT Filed: Dec. 8, 2023
(86) PCT No.: PCT/KR2023/020197

§ 371 Date: Aug. 1, 2025

(30) Foreign Application Priority Data
Feb. 3, 2023 (KR) 10-2023-0014824

(51) Int. Cl. G06T 17/00; A61B 6/51; G06F 30/27; G06T 7/11; G06T 19/20

(52) U.S. Cl. CPC G06T 17/00; A61B 6/51; G06F 30/27; G06T 7/11; G06T 19/20; G06T 2200/04; G06T 2207/10116; G06T 2207/20084; G06T 2207/30036; G06T 2210/41; G06T 2219/2016

(57) ABSTRACT

Disclosed is a device for generating a three-dimensional tooth model, including a memory including instructions and a processor for processing the instructions, where the memory and the processor are connected. When the instructions are executed by the processor, the processor: receives a radiographic medical image in which a plurality of teeth of a user are displayed; uses a first neural network model using the radiographic medical image as an input, and thereby acquires a first tooth model in which the plurality of teeth are divided; acquires patch images respectively corresponding to the plurality of teeth from the radiographic medical image on the basis of the divided teeth in the first tooth model; and uses a second neural network using each of the patch images as inputs, and thereby acquires a second tooth model in which each tooth is divided into the enamel, dentine, and pulp.

### TECHNICAL FIELD

The following embodiments relate to a three-dimensional (3D) tooth model generation method and device.

### BACKGROUND ART

Recently, research on image and video segmentation technology using neural network models is actively being conducted based on various data and structures. Representative neural network models include a convolutional neural network (CNN) model, a recurrent neural network (RNN) model, a long short-term memory model, and an attention mechanism-based transformer model.

Segmentation technology using neural network models in medical images is being used in various medical images, such as brain tumor diagnosis systems and deep learning-based body organ segmentation. In cone-beam computed tomography (CBCT), the importance of an artificial intelligence-based three-dimensional (3D) tooth segmentation system in the oral and maxillofacial area is also increasing, and the system may contribute to the diagnosis and prognosis of dental caries, tooth fracture, and dental root canal mutation patterns.

### DISCLOSURE OF THE INVENTION

### Technical Solutions

A three-dimensional (3D) tooth model generation device according to an embodiment includes a memory including instructions and a processor connected to the memory and configured to execute the instructions, wherein, when the instructions are executed by the processor, the processor is configured to receive a radiographic medical image in which a plurality of teeth of a user is represented, obtain a first tooth model in which the plurality of teeth is segmented, using a first neural network model that uses the radiographic medical image as an input, obtain patch images respectively corresponding to the plurality of teeth from the radiographic medical image based on the segmented teeth represented in the first tooth model, and obtain a second tooth model in which enamel, dentin, and pulp are segmented for each individual tooth, using a second neural network model that uses each of the patch images as an input.

The first tooth model may represent an identification number and a position of each of the plurality of teeth.

The processor may be configured to obtain a second tooth model using the first tooth model, the identification number, and the position.

The first neural network model may include an encoder and a decoder based on a 3D convolutional neural network (CNN).

The first neural network model may be trained to adjust parameters of the first neural network model using a cross-entropy loss function and Dice loss function.

The processor may be configured to downsample the radiographic medical image and provide the downsampled radiographic medical image to the first neural network model.

A 3D tooth model generation method performed by a 3D tooth model generation device according to an embodiment includes receiving a radiographic medical image in which a plurality of teeth of a user is represented, obtaining a first tooth model in which the plurality of teeth is segmented, using a first neural network model that uses the radiographic medical image as an input, obtaining patch images respectively corresponding to the plurality of teeth from the radiographic medical image based on the segmented teeth represented in the first tooth model, and obtaining a second tooth model in which enamel, dentin, and pulp are segmented for each individual tooth, using a second neural network model that uses each of the patch images as an input.

The first tooth model may represent an identification number and a position of each of the plurality of teeth.

The obtaining of the second tooth model may include obtaining a second tooth model using the first tooth model, the identification number, and the position.

The first neural network model may include an encoder and a decoder based on a 3D CNN.

The first neural network model may be trained to adjust parameters of the first neural network model using a cross-entropy loss function and Dice loss function.

The obtaining of the first tooth model may include downsampling the radiographic medical image and providing the downsampled radiographic medical image to the first neural network model.

### BRIEF DESCRIPTION OF DRAWINGS

FIG. 1 is a diagram illustrating a tooth structure.

FIG. 2 is a flowchart illustrating a three-dimensional (3D) tooth model generation method according to an embodiment.

FIG. 3 is a diagram illustrating a learning process of a first neural network model, according to an embodiment.

FIG. 4 is a diagram illustrating a process of obtaining patch images, according to an embodiment.

FIG. 5 is a diagram illustrating an operation of a second neural network model, according to an embodiment.

FIG. 6 is a diagram illustrating a process of obtaining a second tooth model, according to an embodiment.

FIG. 7 is a diagram illustrating a configuration of a 3D tooth model generation device according to an embodiment.

### BEST MODE FOR CARRYING OUT THE INVENTION

The following detailed structural or functional description is provided as an example only and various alterations and modifications may be made to the embodiments. Thus, an actual form of implementation is not construed as limited to the embodiments described herein and should be understood to include all changes, equivalents, and replacements within the idea and the technical scope of the disclosure.

Although terms such as first, second, and the like are used to describe various components, the components are not limited to the terms. These terms should be used only to distinguish one component from another component. For example, a first component may be referred to as a second component, and similarly, the second component may also be referred to as the first component.

It should be noted that when one component is described as being “connected,” “coupled,” or “joined” to another component, the first component may be directly connected, coupled, or joined to the second component, or a third component may be between the first and second components.

The singular forms “a,” “an,” and “the” used herein are intended to include the plural forms as well, unless the context clearly indicates otherwise. It will be further understood that the terms “comprises/comprising” and/or “includes/including,” when used herein, specify the presence of stated features, integers, steps, operations, elements, and/or components, but do not preclude the presence or addition of one or more other features, integers, steps, operations, elements, components and/or groups thereof.

Unless otherwise defined, all terms used herein including technical and scientific terms have the same meanings as those commonly understood by one of ordinary skill in the art to which this disclosure pertains. Terms such as those defined in commonly used dictionaries are to be interpreted as having a meaning that is consistent with their meaning in the context of the relevant art and are not to be interpreted in an idealized or overly formal sense unless expressly so defined herein.

Hereinafter, the embodiments are described in detail with reference to the accompanying drawings. When describing the embodiments with reference to the accompanying drawings, like reference numerals refer to like components and a repeated description related thereto is omitted.

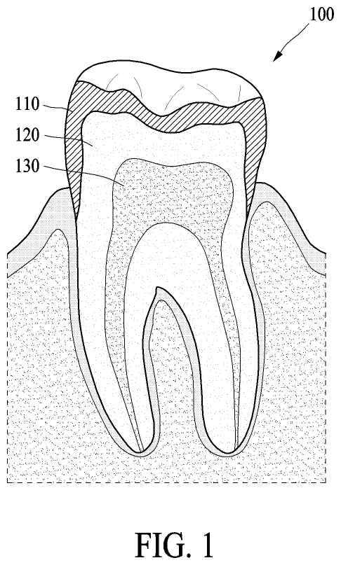

FIG. 1 is a diagram illustrating a tooth structure.

Referring to FIG. 1, a tooth 100 of a human, according to an embodiment, may be structurally divided into enamel 110, dentin 120, and pulp 130. The enamel 110 may be located on top of the tooth 100 and may represent a shape and outline of a crown. The enamel 110 may be exposed to an oral cavity. The dentin 120 may be located beneath the enamel 110. Hardness of the dentin 120 may be lower than hardness of the enamel 110 and higher than hardness of bone. The pulp 130 may be in a pulp cavity of the tooth 100 and may be located beneath the dentin 120. The pulp 130 may include nerves and blood vessels.

Typically, a person may have about 32 teeth, each of which has a structure like the tooth 100 illustrated in FIG. 1. Detailed segmentation of an internal structure of the tooth 100 of a human may help medical professionals understand the internal structure of the tooth 100, thereby helping treatment of human teeth.

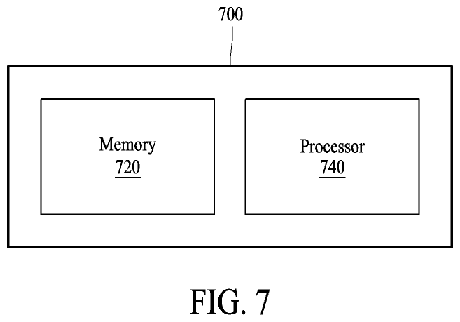

The 3D tooth model generation device described below (e.g., a 3D tooth model generation device 700 of FIG. 7) may generate a 3D tooth model in which, for example, not only an overall shape of the tooth 100 but also detailed internal structures of the tooth 100, such as the enamel 110, the dentin 120, and the pulp 130, are 3D segmented for each of 32 teeth of a human. The 3D tooth model generation device may receive a medical image (e.g., a cone-beam computed tomography (CBCT) image) showing a plurality of teeth of a user, and using the medical image and two different neural network models, may obtain a 3D tooth model in which the enamel 110, the dentin 120, and the pulp 130 are segmented for each of the human teeth. A medical professional treating the teeth of the user may more accurately and clearly recognize a structure of the teeth of the user by using the 3D tooth model obtained from the 3D tooth model generation device and thereby effectively treat the teeth of the user.

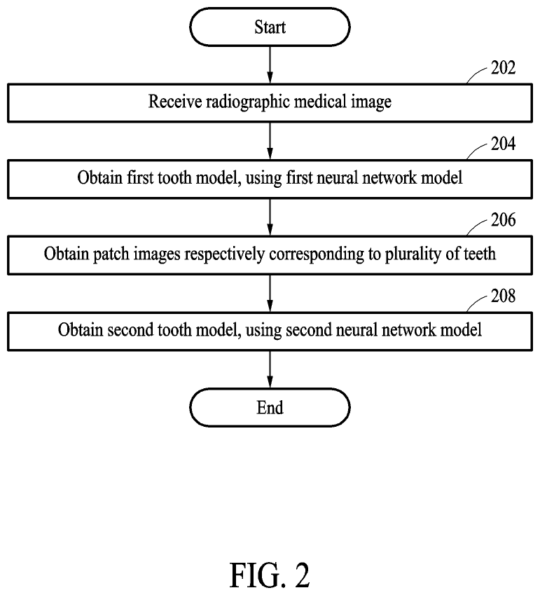

FIG. 2 is a flowchart illustrating a 3D tooth model generation method according to an embodiment. For example, operations of the 3D tooth model generation method may be performed by a 3D tooth model generation device (e.g., the 3D tooth model generation device 700 of FIG. 7). Some operations of FIG. 2 may be performed simultaneously or in parallel to other operations, and the order of the operations may change. In addition, some of the operations may be omitted, or another operation may be additionally performed.

In operation 202, the 3D tooth model generation device may receive a radiographic medical image. The radiographic medical image may be, for example, a computed tomography (CT) image, and the CT image may be, for example, a CBCT image or a spiral CT image. The type of radiographic medical image used by the 3D tooth model generation device is not limited thereto and may vary depending on the embodiment. The radiographic medical image may represent a medical image of a plurality of teeth of a user taken using a radiographic device.

In operation 204, the 3D tooth model generation device may obtain a first tooth model by using a first neural network model. The 3D tooth model generation device may obtain the first tooth model, in which the plurality of teeth is segmented, by using the first neural network model that uses a radiographic medical image as an input. The first neural network model may correspond to a U-Net model including an encoder and a decoder based on a 3D convolutional neural network (CNN). The U-Net model may refer to a model based on an end-to-end fully convolutional network (FCN) proposed for the purpose of image segmentation in the medical field. The first tooth model may be a tooth model that models external shapes of all teeth of the user in 3D.

Each of the encoder and decoder of the first neural network model may have a hierarchical structure including a plurality of (e.g., five) layers. The 3D tooth model generation device may, using the encoder of the first neural network model, analyze a radiographic medical image and extract features of each tooth from the radiographic medical image. A 3D tooth model generation device may, using the decoder of the first neural network model, generate the first tooth model by using the features extracted through the encoder.

The first neural network model may extract an outline of each one of all teeth of the user. The first tooth model may represent an identification number and a position of each of the plurality of teeth represented in the radiographic medical image. When the radiographic medical image is input, the first neural network model may identify types and positions of the teeth represented in the radiographic medical image and determine the identification number and tooth position for each of the teeth of the user based on identification results.

The first neural network model may be a neural network model trained to extract outlines of the teeth represented in the radiographic medical image based on the input radiographic medical image and to output the outlines as a 3D model. During a learning process, a training image (e.g., the radiographic medical image) may be input to the first neural network model, and the first neural network model may estimate the outlines, types, and positions of the teeth based on the input training image. The first neural network model may output, based on the estimated information, a 3D tooth model for the outline of each of all the teeth included in the training image.

A difference between the 3D tooth model output by the first neural network model and a 3D tooth model (which functions as a ground truth (GT)) corresponding to the training image used for learning may be calculated. In addition, during the learning process, parameters (e.g., biases and connection weights between artificial neurons) of the first neural network model may be updated so that the difference may be reduced. The difference may be calculated, for example, by using various well-known loss functions. During the learning process, the above process may be repeatedly performed for a large number of training images, and the parameters of the first neural network model may be optimized through the learning process.

In operation 206, the 3D tooth model generation device may obtain patch images respectively corresponding to the plurality of teeth of the user from the radiographic medical image. Each patch image may represent an image of one tooth among the plurality of teeth. According to an embodiment, there may be multiple radiographic medical images of the teeth of the user, and the 3D tooth model generation device may extract patch images of a same tooth from each of the radiographic medical images. Through this process, a plurality of patch images may be extracted for each of the teeth of the user.

In operation 208, the 3D tooth model generation device may obtain a second tooth model in which enamel, dentin, and pulp are segmented for each tooth by using a second neural network model that uses each of the patch images as an input. The 3D tooth model generation device may obtain the second tooth model using the first tooth model, the identification number assigned to each of the plurality of teeth represented by the radiographic medical image, and a position of each of the plurality of teeth represented by the radiographic medical image.

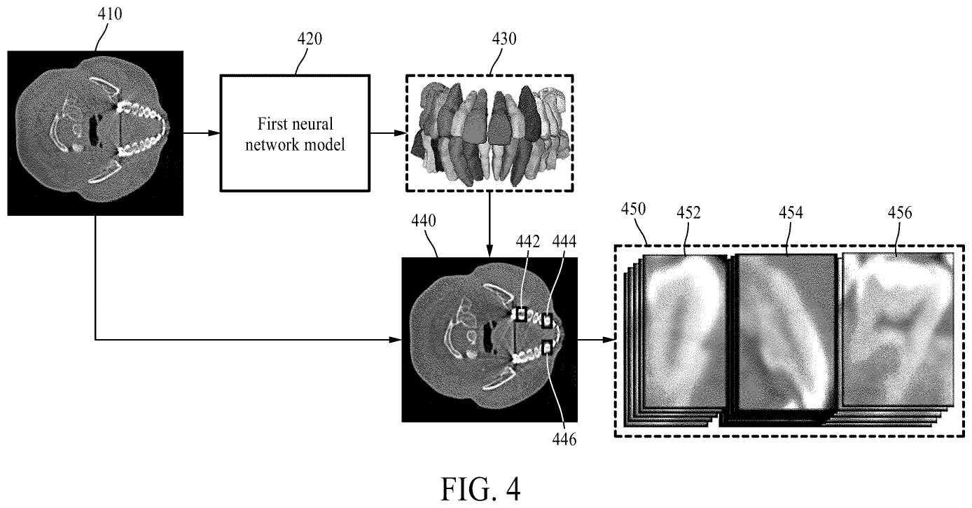

The second neural network model may be a neural network model trained to extract an enamel portion, a dentin portion, and a pulp portion of a tooth represented in the patch image based on the input patch image and to output a 3D individual tooth model in which the tooth is segmented into enamel, dentin, and pulp. During the learning process, patch images (e.g., patch images 450 of FIG. 4) may be input to the second neural network model, and the second neural network model may estimate the position and outline of the enamel, dentin, and pulp of the tooth represented in the patch image based on the input patch image. The second neural network model may output, based on the estimated information, the 3D individual tooth model in which the enamel, dentin, and pulp are segmented for the teeth included in the patch image.

During the learning process of the second neural network model, a difference between the 3D individual tooth model output by the second neural network model and a 3D tooth model (which functions as a GT) corresponding to the training image used for learning may be calculated. In addition, parameters of the second neural network model may be updated so that the difference may be reduced. The difference may be calculated, for example, by using various well-known loss functions. During the learning process, the above process may be repeatedly performed for a large number of training images, and the parameters of the second neural network model may be optimized through the learning process.

The 3D individual tooth model obtained from the second neural network model may represent an identification number corresponding to the tooth represented in the 3D individual tooth model. The identification number of each of the teeth represented in the plurality of 3D individual tooth models obtained from the second neural network model may correspond to one of the identification numbers for each of the teeth represented in the first tooth model obtained from the first neural network model.

The 3D tooth model generation device may obtain the second tooth model, using the first tooth model and the identification number and position of each of the plurality of teeth represented in the first tooth model. For example, the 3D tooth model generation device may confirm, based on the first tooth model, the identification number of a tooth represented in a 3D individual tooth model obtained from the second neural network model and an identification number corresponding to the identification number among the plurality of teeth represented in the first neural network model, and may replace a 3D tooth model at the corresponding position with the 3D individual tooth model. The 3D tooth model generation device may obtain a 3D individual tooth model (corresponding to the second tooth model) in which enamel, dentin, and pulp are segmented for each of the plurality of teeth represented in the radiographic medical image by performing the above process on each of the plurality of teeth represented in the first tooth model.

In a case of individual teeth represented in the radiographic medical image, distinction between enamel, dentin, and pulp is not clear so medical professionals have difficulty in determining the internal structure of the teeth of a user. The 3D tooth model generation device may provide a 3D tooth model (corresponding to a second tooth model) in which enamel, dentin, and pulp are clearly distinguished for each individual tooth of the user obtained from a radiographic medical image, and a medical professional may clearly recognize a boundary between the enamel, dentin, and pulp for each tooth of the user through the 3D tooth model generated by the 3D tooth model generation device, which may be of great help in dental treatment.

In addition, the 3D tooth model generation device may obtain the second tooth model through two stages using the first neural network model and the second neural network model, which may reduce the amount of data processed by each neural network model and reduce the overall processing time required to obtain the second tooth model.

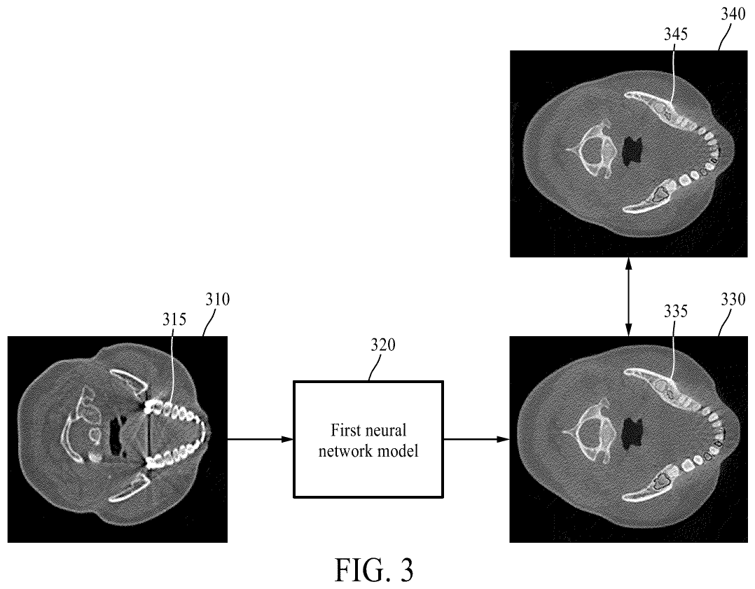

FIG. 3 is a diagram illustrating a learning process of a first neural network model, according to an embodiment.

Referring to FIG. 3, a first neural network model 320 may receive a radiographic medical image 310. A processor (e.g., a processor 740 of FIG. 7) of a 3D tooth model generation device (e.g., the 3D tooth model generation device 700 of FIG. 7) may downsample the radiographic medical image 310 and provide the downsampled radiographic medical image 310 to the first neural network model 320. The processor may reduce an amount of data processed by the first neural network model 320 and increase a processing speed of the first neural network model 320 by downsampling the radiographic medical image 310.

The radiographic medical image 310 may represent a plurality of teeth 315. The first neural network model 320 may be a model trained to receive a two-dimensional (2D) radiographic medical image representing a plurality of teeth, segment each of the plurality of teeth, and output a tooth model representing an identification number and a position for each of the plurality of teeth. The first neural network model 320 may output a predicted image 330, in which each of the plurality of teeth 315 represented in the radiographic medical image 310 is segmented. The predicted image 330 may represent an identification number and a position of each of a plurality of teeth 335.

The first neural network model 320 may be trained through a process of comparing a training image (which functions as a GT) 340, in which each of the plurality of teeth 315 represented in the radiographic medical image 310 is segmented, with the predicted image 330 output by the first neural network model 320. The 3D tooth model generation device may adjust parameters of the first neural network model 320 using a loss function based on a difference value between the training image 340 and the predicted image 330. The 3D tooth model generation device may adjust the parameters of the first neural network model 320 based on a loss function by using, for example, a back propagation algorithm. The first neural network model 320 may be trained using, for example, a cross-entropy loss function and the Dice loss function, but the type of loss function used for training the first neural network model 320 is not limited thereto and may vary depending on the embodiment.

During the learning process of the first neural network model 320, a data augmentation method may have been used to increase the amount of training data to obtain a high-reliability model. For example, during the learning process of the first neural network model 320, random flip, random rotation, and random affine transformation may have been applied to the training data, but the type of data augmentation method is not limited thereto.

FIG. 4 is a diagram illustrating a process of obtaining patch images, according to an embodiment.

Referring to FIG. 4, a first neural network model 420 may receive a radiographic medical image 410. The first neural network model 420 may output a first tooth model 430, in which each of a plurality of teeth represented in the radiographic medical image 410 is segmented, based on the radiographic medical image 410. A first tooth model 430 may represent an identification number and a position of each of the plurality of teeth represented in the radiographic medical image 410. The first neural network model 420 may output the first tooth model 430, which is a 3D image, from the radiographic medical image 410, which is a 2D image.

A 3D tooth model generation device (e.g., the 3D tooth model generation device 700 of FIG. 7) may obtain, from the radiographic medical image 410, the patch images 450 respectively corresponding to the plurality of teeth, based on the segmented teeth represented in the first tooth model 430. For example, among the patch images 450, a patch image 452 may be a patch image corresponding to a tooth 442 among a plurality of teeth represented in a radiographic medical image 440 (corresponding to the radiographic medical image 410), a patch image 454 may be a patch image corresponding to a tooth 444, and a patch image 456 may be a patch image corresponding to a tooth 446. The patch images 450 may be transmitted to a second neural network model.

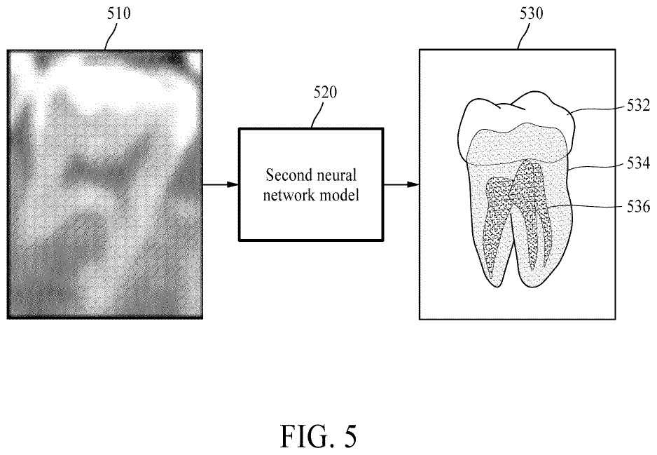

FIG. 5 is a diagram illustrating an operation of a second neural network model, according to an embodiment.

Referring to FIG. 5, a second neural network model 520 may receive a patch image 510. The patch image 510 may be a patch image corresponding to one of a plurality of teeth represented in a radiographic medical image (e.g., the radiographic medical image 440 of FIG. 4). The second neural network model 520 may be a model trained to receive a 2D patch image including a tooth image and generate a 3D individual tooth model, in which enamel, dentin, and pulp are segmented, for the tooth image.

The second neural network model 520 may output a 3D individual tooth model 530 from the patch image 510. The 3D individual tooth model 530 may be a 3D tooth model in which enamel 532, dentin 534, and pulp 536 are segmented for a tooth included in the patch image 510. The 3D individual tooth model 530 may represent an identification number corresponding to a tooth represented in the patch image 510. The identification number represented by the 3D individual tooth model 530 may correspond to one of a plurality of teeth indicated in a first tooth model (e.g., the first tooth model 430 of FIG. 4). The second neural network model 520 may receive patch images respectively corresponding to the plurality of teeth included in the radiographic medical image and output the 3D individual tooth model 530, in which enamel, dentin, and pulp are segmented for each of the teeth respectively corresponding to the patch images.

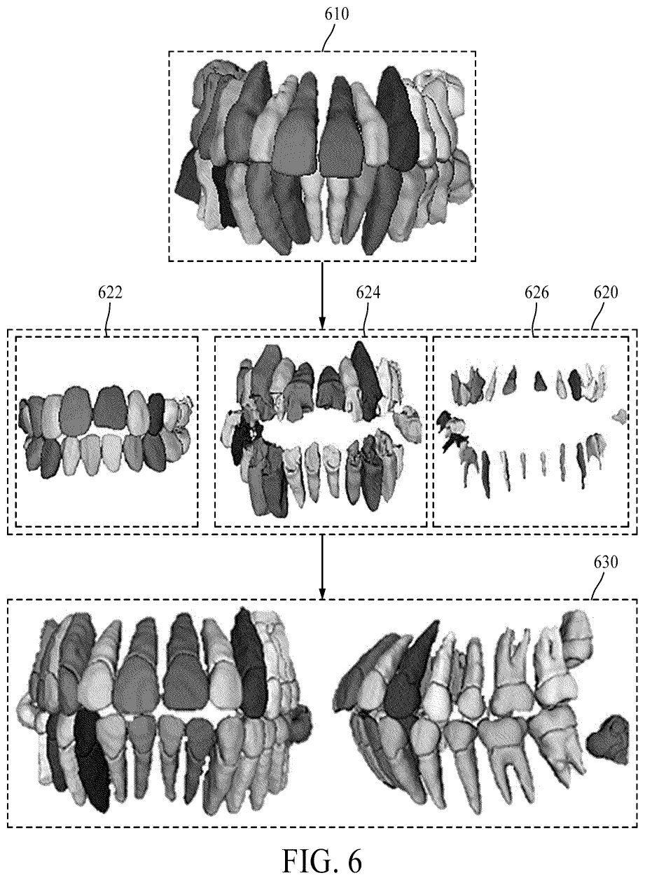

FIG. 6 is a diagram illustrating a process of obtaining a second tooth model, according to an embodiment.

A 3D tooth model generation device (e.g., the 3D tooth model generation device 700 of FIG. 7) may obtain a first tooth model 610, in which each of a plurality of teeth represented in a radiographic medical image is segmented, using a first neural network model. The first tooth model 610 may represent an identification number and a position of each of the plurality of teeth. The 3D tooth model generation device may, using radiographic medical images, obtain patch images respectively corresponding to a plurality of teeth represented in the radiographic medical images. The 3D tooth model generation device may provide each of the patch images to a second neural network model. The second neural network model may receive the patch images as an input and output 3D individual tooth models in which enamel, dentin, and pulp are segmented for the teeth respectively corresponding to the patch images.

The 3D tooth model generation device may obtain a tooth model in which enamel 622, dentin 624, and pulp 626 are segmented for each of the plurality of teeth. The 3D tooth model generation device may finally obtain a second tooth model 630 in which the enamel 622, the dentin 624, and the pulp 626 are segmented for each of the plurality of teeth represented in the radiographic medical image, using the first tooth model 610 and the identification number and the position of each of the plurality of teeth represented in the first tooth model 610.

FIG. 7 is a diagram illustrating a configuration of a 3D tooth model generation device according to an embodiment.

Referring to FIG. 7, the 3D tooth model generation device 700 may include a memory 720 including instructions, and the processor 740 for executing the instructions.

The processor 740 may control other components (e.g., hardware or software components) of the 3D tooth model generation device 700 and perform various data processing or operations. As at least a portion of the data processing or operations, the processor 740 may store, in the memory 720, instructions or data received from another component, process instructions or data stored in the memory 720, and store result data in the memory 720. Operations performed by the processor 740 may be substantially the same as the operations of the 3D tooth model generation device 700.

The memory 720 may store information necessary for the processor 740 to perform a processing operation. For example, the memory 720 may store instructions executed by the processor 740 and store related information while software or a program is executed in the 3D tooth model generation device 700. The memory 720 may include volatile memory such as random-access memory (RAM), dynamic RAM (DRAM), and static RAM (SRAM), and/or non-volatile memory known in the art such as flash memory.

The processor 740 may receive a radiographic medical image that represents a plurality of teeth of a user. The radiographic medical image may be, for example, a CBCT image. The processor 740 may provide a radiographic medical image to a first neural network model. The processor 740 may downsample the radiographic medical image and provide the downsampled radiographic medical image to the first neural network model. The processor 740 may reduce an amount of data processed by the first neural network model and increase a data processing speed of the first neural network model by downsampling the radiographic medical image.

The processor 740 may obtain a first tooth model in which a plurality of teeth is segmented, using the first neural network model that uses the radiographic medical image as an input. The first neural network model may include an encoder and a decoder based on a 3D CNN. The first neural network model may, using an encoder, extract features of the teeth represented in the radiographic medical image from the radiographic medical image. The first neural network model may, using a decoder, output a first tooth model based on the extracted features. The first neural network model may be trained to adjust parameters of the first neural network model using a cross-entropy loss function and the Dice loss function, but the loss function used by the first neural network model is not limited thereto and may vary depending on the embodiment.

The first tooth model may include respective identification numbers and positions of the plurality of teeth. The processor 740 may obtain patch images respectively corresponding to the plurality of teeth from the radiographic medical image based on the segmented teeth represented in the first tooth model. The processor 740 may obtain a second tooth model, in which enamel, dentin, and pulp are segmented for each individual tooth, using a second neural network model that uses each of the patch images as an input. The processor may obtain the second tooth model, using the first tooth model and the respective identification numbers and positions of the plurality of teeth.

The embodiments described herein may be implemented using a hardware component, a software component, and/or a combination thereof. The devices, methods, and components described in the embodiments may be implemented using a general-purpose computer or a special-purpose computer, such as, for example, a processor, a controller, an arithmetic logic unit (ALU), a digital signal processor (DSP), a microcomputer, a field-programmable gate array (FPGA), a programmable logic unit (PLU), a microprocessor, or any other device capable of responding to and executing instructions. The processing device may run an operating system (OS) and software applications that run on the OS. The processing device may also access, store, manipulate, process, and create data in response to execution of the software. For purpose of simplicity, the processing device is described as singular. However, one of ordinary skill in the art will appreciate that a processing device may include multiple processing elements and/or multiple types of processing elements. For example, the processing device may include a plurality of processors, or a single processor and a single controller. In addition, a different processing configuration is possible, such as one including parallel processors.

The software may include a computer program, a piece of code, instructions, or one or more combinations thereof, to independently or collectively instruct or configure the processing device to operate as desired. The software and/or data may be stored in any type of machine, component, physical or virtual equipment, or computer storage medium or device for the purpose of being interpreted by the processing device or providing instructions or data to the processing device. The software may also be distributed over network-coupled computer systems so that the software is stored and executed in a distributed fashion. The software and data may be stored in a non-transitory computer-readable recording medium. The methods according to the embodiments may be recorded in non-transitory computer-readable media including program instructions to implement various operations of the embodiments. The media may also include the program instructions, data files, data structures, and the like alone or in combination. The program instructions recorded on the media may be those specially designed and constructed for the purposes of embodiments, or they may be of the kind well-known and available to one of ordinary skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as floptical disks; and hardware devices that are specially configured to store and perform program instructions, such as ROM, RAM, flash memory, and the like. Examples of program instructions include both machine code, such as those produced by a compiler, and files containing high-level code that may be executed by the computer using an interpreter. The above-described hardware devices may be configured to act as one or more software modules in order to perform the operations of the examples, or vice versa.

While the embodiments are described with reference to a limited number of drawings, it will be apparent to one of ordinary skill in the art that various alterations and modifications in form and details may be made in these embodiments without departing from the spirit and scope of the claims and their equivalents. For example, suitable results may be achieved if the described techniques are performed in a different order and/or if components in a described system, architecture, device, or circuit are combined in a different manner and/or replaced or substituted by other components or their equivalents.

1. A three-dimensional (3D) tooth model generation device comprising:
a memory comprising instructions; and
a processor connected to the memory and configured to execute the instructions,
wherein, when the instructions are executed by the processor, the processor is configured to:
receive a radiographic medical image in which a plurality of teeth of a user is represented;
obtain a first tooth model in which the plurality of teeth is segmented, using a first neural network model that uses the radiographic medical image as an input;
obtain patch images respectively corresponding to the plurality of teeth from the radiographic medical image based on the segmented teeth represented in the first tooth model; and
obtain a second tooth model in which enamel, dentin, and pulp are segmented for each individual tooth, using a second neural network model that uses each of the patch images as an input.

2. The 3D tooth model generation device of claim 1, wherein the first tooth model represents an identification number and a position of each of the plurality of teeth.

3. The 3D tooth model generation device of claim 2, wherein the processor is configured to:
obtain a second tooth model using the first tooth model, the identification number, and the position.

4. The 3D tooth model generation device of claim 1, wherein the first neural network model comprises:
an encoder and a decoder based on a 3D convolutional neural network (CNN).

5. The 3D tooth model generation device of claim 1, wherein the first neural network model is trained to adjust parameters of the first neural network model using a cross-entropy loss function and Dice loss function.

6. The 3D tooth model generation device of claim 1, wherein the processor is configured to:
downsample the radiographic medical image and provide the downsampled radiographic medical image to the first neural network model.

7. A three-dimensional (3D) tooth model generation method performed by a 3D tooth model generation device comprising:
receiving a radiographic medical image in which a plurality of teeth of a user is represented;
obtaining a first tooth model in which the plurality of teeth is segmented, using a first neural network model that uses the radiographic medical image as an input;
obtaining patch images respectively corresponding to the plurality of teeth from the radiographic medical image based on the segmented teeth represented in the first tooth model; and
obtaining a second tooth model in which enamel, dentin, and pulp are segmented for each individual tooth, using a second neural network model that uses each of the patch images as an input.

8. The tooth model generation method of claim 7, wherein the first tooth model represents an identification number and a position of each of the plurality of teeth.

9. The tooth model generation method of claim 8, wherein the obtaining of the second tooth model comprises:
obtaining a second tooth model using the first tooth model, the identification number, and the position.

10. The tooth model generation method of claim 7, wherein the first neural network model comprises:
an encoder and a decoder based on a 3D convolutional neural network (CNN).

11. The tooth model generation method of claim 7, wherein the first neural network model is trained to adjust parameters of the first neural network model using a cross-entropy loss function and Dice loss function.

12. The tooth model generation method of claim 8, wherein the obtaining of the first tooth model comprises:
downsampling the radiographic medical image and providing the downsampled radiographic medical image to the first neural network model.

13. A non-transitory computer-readable storage medium storing instructions that, when executed by a processor, cause the processor to perform the method of claim 7.
